(19) United States
(12) Patent Application Publication
(10) Pub. No.: US 2003/0144561 A1
(43) Pub. Date: Jul. 31, 2003

(54) Method for preparing a hydrofluoroalkane and catalyst

(76) Inventors: Vincent Wilmet, Louvain-La-Neuve (BE); Georges Lejeune, Dolhain-Limbourg (BE)

Correspondence Address: CONNOLLY BOVE LODGE & HUTZ, LLP, 1220 N MARKET STREET, P O BOX 2207, WILMINGTON, DE, 19899

(21) Appl. No.: 10/257,453

(22) PCT Filed: Apr. 5, 2001
(86) PCT No.: PCT/EP01/03888

(30) Foreign Application Priority Data
Apr. 12, 2000 (FR) 00/05355

(51) Int. Cl. C07C 19/08; B01J 23/00

(52) U.S. Cl. 570/169; 502/306; 502/304; 502/311; 502/319

(57) ABSTRACT

Process for the preperation of a hydrodhlorofluoroalkane according to which a halogenated precursor of the hydrochlorofluoroalkane is reated with hydrogen fluoride in the presence of a catalyst comprising chromiun (Cr) and another metal.

The present invention relates to a process for the preparation of a hydrochlorofluoroalkane and a catalyst.

HydrochlorofluoroaLksnes are of interest in partic as reaction intermediate for the manufacture of hydrofluoroalkanes and for the sythesis of functionalized fluorinated orgainc compounds of high added value which can be for example, in the pharmaceutical industry.

When a halogenated precursor of a hydrochlorofluoroalkane is reacted in the presence of hydrogen fluoride under fluorination conditions, it is difficult to avoid the formation of overfluorinated products.

It is, for example, mown from EPA-957 074 to carry out a hydrofluorination of perchloroethylene with hydrogen fluoride in the presence of a chromium/zirconium catalyst in which the Zr/Cr atonmc ratio envisaged does not exceed 0.11. This known process produces considerable amounts of 1,1,1,2-tetrachloro-2fluoroethane (HCFTC124) and of pentagluoroethane (EFC125), which are not desirable in a synthesis of 1,1,1-trichloro-2,2-difluoroethane (HCFC-123).

It was therefore desirable to find a process which would make possible access in a controlled and selective manner to a specific hydrochlorofluoroalkane while minimizing the formation of unwanted chlorofluoroalkanes.

The invention consequently relates to a process for the preparation of a hydrochlorofluoroalkane according to which a halogenated precusor of the hydrochlorofluoroaluae is reacted with hydrogen fluoride in the presence of a catalyst comprising chromium (Cr) and another metal (M, with an M/Cr atomic ratio of at least 0.2.

It has been found, surprisingly, that the process according to the invention makes possible access with high selectvity to a specific hydrochlorofluoroalkane with high overall selecty for hydrochlorfluoroalkane and for hydrochlorofluorinated precursors of the said hydrochlorofluoroalkane.

In the process according to the invention, the M/Cr atomic ratio in the catalyst is often at least 0.3. It is preferably at least 0.5. Generally, the M/Cr atomic ratio is at most 100. It is usually at most 10.

In the process according to the invention, the metal M can in particular be barium, bismuth, calcium, cerium, cobalt, copper, iron, manganese, nickel, vanadium, zinc and/or zirconium. The metel M is preferably selected from the group consisting of barium, bismuth, calcium, cerium and zirconium. In a very particularly preferred way, the metal M is zirconium.

The catalyst can be a bulk or supported catalyst. A bulk catalyst is preferred.

The catalyst generally exhibits a specific surface, determined according to the BET method with nitrogen, of at least 10 m²/g, prefereably of at least 15 m²/g. The specific surface is generally at most 200 m²/g. It is preferably at most 100 m²/g.

In the process accord to the invention, the catalyst is preferably obtained by fluorination of a mixed oxide of chromium and of metal M. The fluorination is preferably carried out with hydrogen fluoride, optionally diluted with an inert gas, such as nitrogen or helilm. The duration of the fluorination is generally from 1 to 100 h. The fluorination can be carried out, and 400° C. It is prersbly at most 350° C. The fluorination can be carried out, for example, immediately before the reaction of the balogenated precursor with hydrogen fluoride, prerably in the reactor used for the later reaction.

The preperation of the mixed oxide preferably compnses a stage of coprecipitation by reaction of an aqueous solution of soluble metal and chromium salt, which is reacted with an aqueous ammonia solution.

The preparation of the mixed oxide can advantageously comprise one or more drying or calcination stages. The temperature of the calcination is generally from 150° C. to 400° C. The calcination temperate is preferably at most 350° C. A calcination temperature of at most 340° C. is more particulary preferred. The calcination is often carried out at a temperature of at least 200° C. After calcinaton, the mixed oxide generally exhibits a specific surface, deteried according to the BET method with nitrogen, of at least 150 m/²/g, preferably of at least 180 m²/g. The specific surface is generally at most 350 m²/g. Advantageously, the preparation of the mired oxide comprises a treatment intended to reduce, preferably to essentially completely remove, the content of ammonium ions in the mixed oxide before subjecting it to the fluorintion stage, so that the catalyst includes at most 1% by weight of ammonium ions. It preferably exhibits a content of ammonium ions of at most 0.5% by weight. The content of ammonium ions in the catalyst is preferably at most 0.2% by weight. Excellent results are obtained with a catalyst for which the content of ammonium ions is at most 0.1% by weight. Particularly advantageous results are obtained with a catalyst for which the content of ammonium ions is at most 0.05% by weight.

In the process according to the inventon, the reaction between the hydrogen fluoride and the halogenated precursor usually takes place at a temperature of 150 to 450° C., prefrably in the gas phase. The pressure of the reaction is usually from 0.5 to 30 bar. The molar ratio of the hydrogen fluoride to the halogeated precursor is usually from 1 to 100. The residence the is usually from 1 to 1000 s.

It the process according to the invention, the hydrochlorofluoroalkane obtained is, for example, 2-chloro-1,1,1-trifluoroethane, 2,2-chloro-1,1,1 tri-fluoroethane or 2-chloro-1,1,1,3,3-pentfuoropropane, preferably 2,2-dichloro-1, 1, 1-trifluoroethane. In this case, the halogenated precursor advantageously comprises at least one chlorinated organic compound chosen from perchloroethylene, 1,1,2-tetrachloro-2-fluoroethylene, 1,1,2tichloro-2,2-difluoroethane and 1,1,2,2-tetrachloro-2-fluoroethaue. The chlorinated organic compound is preferably chosen from 1,1,2-trichloro2,2-difluoroethane and perchloroethylene.

The invention also relates to a process for the synthesis of trifluoroacetyl chloride according to which;

(a) the process according to the invention is used to mam re 2,2-dichloro-1,1,1-trifluoroethane; and

(b) a photooxidation ofthe 2,2-dichloro-1,1,1-trifluoroethane obtained is carried out.

The invention also relates to the mixed oxide and to the catalyst which are described above.

Another subject-matter of the invention relates to a method for the preparation of a hydrochlorofluoroalkane, according to which a halogenated precursor of the hydroclorofluoroalkane is reacted with hydrogen floride in the presence of a catalyst comprising chromium (Cr) and bismuth (Bi), which catalyst is preferably composed essentially of chromium and bismuth compounds, in which catalyst the BI/Cr atomic ratio can be less tha or equal to 0.2.

The invention also relates to the said catalyst comprising chromium and bismuth. The Bi/Cr atomic ratio in this catalyst according to the invention is generally at least 0.05, prefersbly at least 0.1. Apart from the atomic ratio, the preparation of the catalyst, its characteristics and the conditions for reaction of the halogenated precursor with hydrogen fluoride in the method according to the invention generally correspond to those described above. The method according to the invention, employiag a catalyst comprising chromium (Cr) and bismuth (Bi), akes possible access with high selectivity to a specific hydrochlorofluoroalkane, with high overall selectivity for hydrochlorofluoroalkane and for hydrochlorofluorinated precursors of the said hydrochlorofluoroalkane.

The examples given below are intended to illustrates the invention without, however, limiting it. In the examples, the degree of conversion of the halogenated precursor is the ratio, expressed as percent, of, on the one hand, the amount employed minus the unconverted amount to, on the other hand, the amount employed; the selectivity for hydro(chloro)fluoroaLkane is the ratio, expressed as percent of the amount of hydro(chloro)fluoroaLane formed to the amount which would have been formed if all the halogenated precursor converted had generated hydro(chloro)fluoroalkane; the overall selectivity is the sum of the selectivity for the desired hydro(chloro)fluoroalkine of all the recoverable intermediates; the yield of hydro(chloro)fluoroalkane is the product of the degree of conversion and the selectivity for this hydro(chloro)fluoroalkane.

### EXAMPLES 1-4

1.61 of an aqueos solution comprising 0.56 mol/l of nitrates of chrornium and of zirconium and exhibiting the desired atomic ratio of the chromium to the zirconium were prepared. 0.71 of aqueous ammonia solution, exhibiting a concentration of NH₄OH of 4 mol, was added to the solution with stirring at ambient temperature. The precipitate was centrifuged. The cake was washed several times with water at approximately 65° C. until at least 80% of the initial content of ammonium ions in the cake, confirmed using Nessler's reagent, had been removed. The washed cake was dried for 2 days at 105° C. The agglomerates obtained after drying were milled to produce grans with a size of less than 5 mm. The grains were subjected to calcination for a total period of 69 h while flushing with nitrogen. The temperature was Rained first at 215° C. and then at 330° C. The specific surface (SS) of the znoed oxide obtained is shown in Table 1. It was detennined according to the BET method with nitrogen, measured on a Carlo Erba Sorptomatico® 1990 device after degassing the samples at ambient temperature for 12 h under a vaccuum of 10⁻⁵ torr.

The mixed oxide was introduced into a tubular reactor made of Hastelloy C. A florination treatment was carried out with hydrogen fluoride gas (10 g/h per 100 cm³ of mixed oxide) mixed wh nitrogen for 8 h at a temperature of 200 to 350° C. The specific surface (SS) of the catayst which ae obtained is shown in Table 1.

10 cm3 of the catalyst and a hydrogea fluoride perchloroethylenm (PER) mixture in a molar ratio of 10 mol/mol were intouced into a tubular reactor with an internal diameter of 15 mm. The reaction pressure was maintained at 1 bar and the temperature at 350° C. The residence time was 12.5 seconds. The results are collated in Table 1 below.

TABLE 1

| | | | | Selectivity | Overall | HCFC- |
|---|---|---|---|---|---|---|
| | Zr/Cr | SS mixed | SS | for HCFC- | selectivity for | 123/HCFC- |
| | atomic | oxide | catalyst | 123 | recyclable | 124 + HFC-125 |
| No. | ratio | (m²/g) | (m²/g) | % | products % | ratio % |
| 1 | 70/90 | 249 | 17 | 30.3 | 93.4 | 8.9 |
| 2 | 50/50 | 261 | 71 | 28.8 | 92.9 | 6.7 |
| 3 | 30/70 | 251 | 53 | 39.5 | 66.6 | 1.4 |
| 4* | 10/90 | 238 | 77 | 12.1 | 12.6 | 0.15 |

*Example not in accordance with the invention.

1. Process for the preparation of a hydrochlorofluorolkaue according to which a halogenated precursor of the hydrochlorofluoroalkane is reached with hydrogen fluoride in the presece of a bulk catalyst comprising chromium (Cr) and another metal (M selected from the group consisting of barium, bismuth, calcium, cerium and zirconium, characterised by an M/Cr atomic ratio at least 0.2.

2. Process accordmg to claim 1, in which the M/Cr aomic ratio is at least 0.3.

3. Process according to claim 2, in which the M/Cr atomic ratio is at least 0.5.

4. Process according to any one of claims 1 to 3, in which the metal M is zirconim.

5. Process according to any one of claims 1 to 4, in which the catalyst exhibits a specific surface, determined according to the BET method, of at least 15 m²/g.

6. Process according to any one of claims 1 to 5, in which the catalyst was obtained by fluorination of a mixed oxide of chromium and of metal M.

7. Process accoring to claim 6, in which the mixed oxide was obtained by coprecipitation of salts of chromium and of metal M.

8. Process accord to claim 6 or 7, in which the mixed oxide exhibits, before fluorination, a specific surface, determined according to the BET method, of at least 150 m²/g.

9. Process according to any one of claims 1 to 8, in which the hydrodhlorofluoroalkane obtained is 2,2dichloro-1,1,1-trifluorothane.

10. Process for the synthesis of trifluoroacetyl chloride, according to which
(a) the process according to any one of claims 1 to 9 is used to manufacture 2,2-dichloro-1,1,1-trifluoroethane;
(b) a photooxidaion of te 2,2-dichlorov1,1,1-trifluoroethane obtained is carried out.

11. Bulk catalyst comprising chromium (Cr) and another metal (M) selected from the group consisting of barium, bismuth, calciums cerium and zircum in an M/Cr atomic ratio of at least 0.2.

12. Mixed oxide comprising chromium (Cr) and another metal M selected from the group consisting of barium, bismuth, calcium, cerium and zirconium in an M/Cr atomic ratio of at least 0.2.
